(19) United States
(12) Patent Application Publication
(10) Pub. No.: US 2004/0077079 A1
(43) Pub. Date: Apr. 22, 2004

(54) Improved in vitro method of culturing mammalian cells for autologous cell implantation/transplantation methods

(76) Inventors: Peter Storgaard, Valby (DK); Kurt Osther, Scottsdale, AZ (US)

Correspondence Address: Peter F Corless, Edwards & Angell, PO Box 9169, Boston, MA, 02209, US

(21) Appl. No.: 10/470,189

(22) PCT Filed: Jan. 29, 2002
(86) PCT No.: PCT/DK02/00065

(30) Foreign Application Priority Data
Jan. 31, 2001 (DK) PA200100162

(51) Int. Cl. C12N 5/08

(52) U.S. Cl. 435/325; 435/366

(57) ABSTRACT

A production method for producing cell colony forming units in vitro from a mammalian tissue explant, the method comprises the steps of a) growing a piece of the mammalian tissue explant in a growth medium to obtain cell colony forming units from immature cells from the piece of explant, and b) harvesting cells from one or more of the cell colony forming units for use in Autologous Cell Implantation/transplantation methods. The mammalian tissue explant is selected from the group consisting of cartilage; bone such as, e.g., bone marrow; connective tissue; muscle tissue such as, e.g., smooth muscle tissue, heart tissue, liver tissue and skeletal muscle tissue; skin tissue such as, e.g., periosteum; mucosal tissue; brain tissue, pancreas tissue and blood vessels. In particularly, the mammalian tissue explant is cartilage, such as elastic, fibro, hyalin or articular hyalin cartilage. The cells obtained are suitable for use in autologous implantation/transplantation methods. In a specific embodiment, the cell obtained are chondroytes, especially for use in autologous chondrocyte implantation (ACI) methods.

### FIELD OF THE INVENTION

The invention relates to methods, kits and production plant for use in the production of one ore more cell colony forming units (CFU) from a mammalian tissue explant and further to the use of cells from such cell colony forming units in therapy, in particular for the autologous cell treatment of a mammal suffering from tissue disorders.

### BACKGROUND OF THE INVENTION

Many mammals such as humans, domestic and/or racing animals suffer from or will suffer from various tissue disorders or tissue related disorders. The tissue may be cartilage; bone such as, e.g., bone marrow; connective tissue; muscle tissue such as, e.g., smooth muscle tissue, heart tissue, liver tissue and skeletal muscle tissue; skin tissue such as, e.g., periosteum; mucosal tissue; brain tissue and pancreas tissue.

With respect to e.g. cartilage tissue more than one million human arthroscopic procedures and total joint replacements are performed each year in the U.S. and Europe together. Included in these numbers are in the U.S., about 90,000 total knee replacements, and around 50,000 procedures for repairing defects in the knee alone per year (In: Praemer, A., Fumer, S., Rice, D. P., Musculoskeletal Conditions in the United. States, Park Ridge, Ill.: American Academy of Orthopaedic Surgeons, 1992, 125).

Human articular cartilage undergoing self-repair is a slow process since many chondrocytes lose their mitotic ability during the first year of life. Defects in articular cartilage, especially in weight-bearing joints, will predictably deteriorate toward osteoarthritis. No conventional method may prevent this deterioration.

Drilling of the subchondral bone can lead to fibrocartilage formation, which is non-resilient and can only be considered a temporary repair that slowly degrades. Animal studies have indicated that introducing proliferated chondrocytes such as articular chondrocytes may reliably reconstruct joint defects (Robinson D., et al., Isr. Med. Assoc. J., 2000, 2:290).

Among different breeds of horses in U.S. and in Europe there are around five million registered “expensive” Thoroughbred Racehorses used in horse races, whereof an estimated 60% is directly or indirectly owned by U.S. horse-owners. Of all breeds Thoroughbreds is the most frequent sufferer of degenerative joint disease, mostly in the form of osteochondritis dissecans (OCD). The most frequent joint affected is the so-called stifle joint (femoropatellar joint, hind leg). The most common age for horses and especially racehorses to develop OCD is between 1 and 6 years old. The earlier, the training of thoroughbreds is started (1 year old or less) the more frequent the disorder will appear.

In general, surgery including arthroscopic intervention is used in most of the cases with clinical symptoms. Around 64% of the horses treated return to their previous use (racing, etc.). Approximately 35% of the horses cannot return to their previous use within racing or may have less possibilities of obtaining previous levels of racing.

A second condition that can be observed, described as OCD is fragmentabon at the back of the fetlock off the proximal or plantar aspect of the first phalanx or long pastern bone. A third, frequent trauma related condition of racehorses is cartilage damage to the cannon bone condyles, which actually is not a true OCD. OCD in shoulder joints often affects large areas of the joint surface and secondary osteoarthritis is common.

Cleaning or resurfacing the cartilage structure for patients have been attempted using subchondral drilling, abrasion, etc. whereby diseased cartilage and even subchondral bone are excised (Insall, J., Clin. Orthop. 1974, 101:61; Ficat, R. P., et al., Clin Orthop. 1979, 144:74; Johnson, L. L., In: (McGinty, J. B., Ed.) Operative Arthroscopy, New York, Raven Press, 1991, 341). However, there is still a need for a method for regenerative treatment or repair of cartilage defects, which also can be performed at an early stage of a joint damage, reducing the number of humans needing future artificial joint replacement surgery. Moreover, it would be an advantage if the method is suitable for use of autologous material, i.e. material from one and the same mammal during the entire process or at least during the critical steps in the process.

Repair of articular cartilage defects may also be performed in other ways than conventional surgery or medical treatment. A relatively new method has been named Autologous Chondrocyte Implantation (ACI) for restoring articular cartilage.

Chondrocyte implantation has proven clinically effective in restoring hyaline-like cartilage to isolated pathological full thickness chondral lesions of the human knee. Several authors have performed chondrocyte implantation in humans with excellent results (Brittberg et al., 1994 New Engi. J. Med.; Minas, T., Am. J. Orthop. 1998, 11:739), (Roberts S., et al., Arthritis & Rheumatism, Vol. 44, no. 11, November 2001, pp 2586-2598).

Methods such as suturing a periostal flap (for instance removed from tibia) over the defect has currently been used either as a treatment procedure in itself, or has been used in combination with implantation of cultured autologous chondrocytes. The methods using this combination, has in principal, been developed by Brittberg et al. Cells cultured using the methods described by Brittberg et al., are used for autologous implantation in knee joints of patients.

### BRIEF DISCLOSURE OF THE INVENTION

The objective of the invention is to provide methods and means to collect a piece of mammalian tissue explant, to produce one or more cell colony forming units (CFU) from a piece of mammalian tissue explant and to use the cell colony forming units in 1) autologous cell therapy, in particular for the treatment of a mammal suffering from tissue disorders and in 2) methods related to a composition in medicine or as a diagnostic tool.

In an aspect, the invention relates to a method for the autologous cell treatment of a mammal suffering from a tissue or tissue related disorder, the method comprising administering to the mammal in need thereof a sufficient amount of one or more cell colony forming units or a composition comprising of one or more cell forming units.

The invention further relates to methods, kits and production plant for use in the production of one ore more cell colony-forming units (CFU) from a mammalian tissue.

Accordingly, in a first aspect the present invention relates to a method of producing cell colony forming units in vitro from a mammalian tissue explant, comprising the steps of stimulating and growing a piece of a mammalian tissue explant in a growth medium to obtain cell colony forming units derived from immature cells from the piece of explant, and harvesting one or more of the cell colony forming units for autologous cell implantation.

In another aspect, the invention relates to one or more cell colony-forming units produced by the method, and which cell colony forming units are related to medicine or may be used as a diagnostic tool.

In a further aspect, the invention relates to a transportation kit for collecting a mammalian tissue explant of a mammal, the kit comprising an instrument for collecting a mammalian tissue explant from a mammalian tissue and a transportation container for preserving and transporting the mammalian tissue explant and, optionally, instructions for the use of the instrument.

In still a further aspect, the invention relates to a delivery kit comprising a container, the container comprising suspended cells derived from one cell colony-forming unit or a group of cell colony forming units in a carrier. Additionally, the delivery kit may contain a cartilage and/or an interface membrane (see e.g. WO 01/06949 for details of such membranes).

In another aspect, the invention relates to a diagnostic method for the determination of the biological activities of a piece of mammalian tissue explant in an in vitro culture system, the method comprising the steps of growing a piece of mammalian tissue explant comprising matrix and cells in a growth medium in a tissue culture flask, and subjecting at least a part of the content of the tissue culture flask to an investigation and analysis to receive information of the nature of the mammalian tissue explant related to the matrix composition and/or information related to the cells derived from the explant and the colony forming units.

In a final aspect, the invention relates to a production plant for the production of cell colony forming units in vitro from a mammalian tissue explant, comprising means for i) application of a piece of the mammalian tissue explant to a tissue culture flask, ii) application of a growth medium to the tissue culture flask, iii) stimulating and growing migrating cells from the piece of mammalian tissue explant into the growth medium, and iv) application of a release medium to the tissue culture flask.

The invention provides novel methods and means for cell isolation and production of one or more cell colony forming units from a piece of tissue explant without the need for using degrading enzymes in the initial cell isolation procedure with the biopsy. The invention provides novel therapeutic methods, which are made in an autologous manner in mammals, for the treatment of tissue disorders.

### DESCRIPTION OF THE DRAWINGS

the invention is further illustrated with reference to the drawing in which

FIG. 1 is a schematic view of an instrument to be used according to the invention to obtain a mammalian tissue explant of well-defined size,

FIG. 2 shows migrant immature cells from cartilage and bone explants, which form colony-forming units of different phenotypes when cultured together. The upper colony to the right is an osteogenic CFU. The lower colony to the left is a chondrogenic CFU,

FIG. 3 shows cell colony-forming units derived from 7 different sources of human cartilage explants,

FIG. 4 shows a cartilage tissue explant obtained by the instrument in FIG. 1. Migrated cells are visible precisely outside of the surface of the cartilage tissue explant,

FIGS. 5a and b show cartilage explants and cell colony-forming units after 4 weeks of culturing after employment of the production plant equipment,

FIG. 6 shows chondrogenic cells obtained from cartilage treated with collagenase. The monolayer culture shows phenotypic changes and fibroblastic morphology after 1 week of culturing,

FIG. 7 shows the periphery of a cell colony-forming unit of immature chondrogenic cells. The immature cells derived from the cell colony were isolated and selected with the cartilage explant system. Cells show chondrogenic phenotypes—some with extensive matrix production,

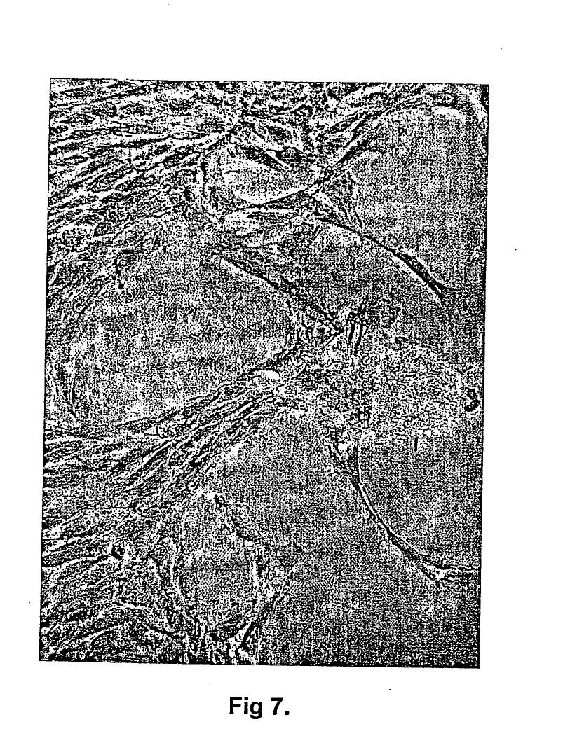

FIG. 8 shows Electron Microscopy (EM) of neocartilage developed in the colony forming units present in the culture flask. The newly formed tissue Is composed of fibrils derived from collagen type II, IX and XI and large aggrecan aggregates composed of hyaluronan, link proteins and proteoglycans. The proteoglycans chains have collapsed during fixation and appear as black spherical globular structures attaching to type II, IX and XI collagen molecules. This EM picture shows that cartilage cells derived from the colony forming units secrete large amount of hyalin cartilage molecules indicating proper differentiation of the cultured chondroblast,

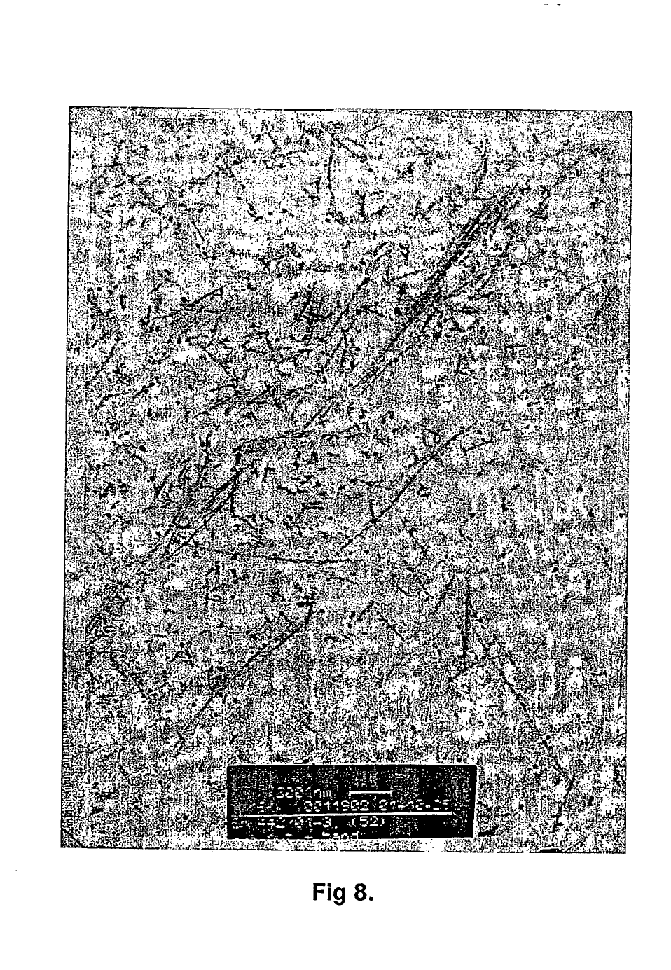

FIG. 9 relates to Example 9. FIG. 9a shows colony forming unit derived from meniscus-explant and FIG. 9b shows the close up of immature cells present in the colony-forming unit derived from a meniscus explant

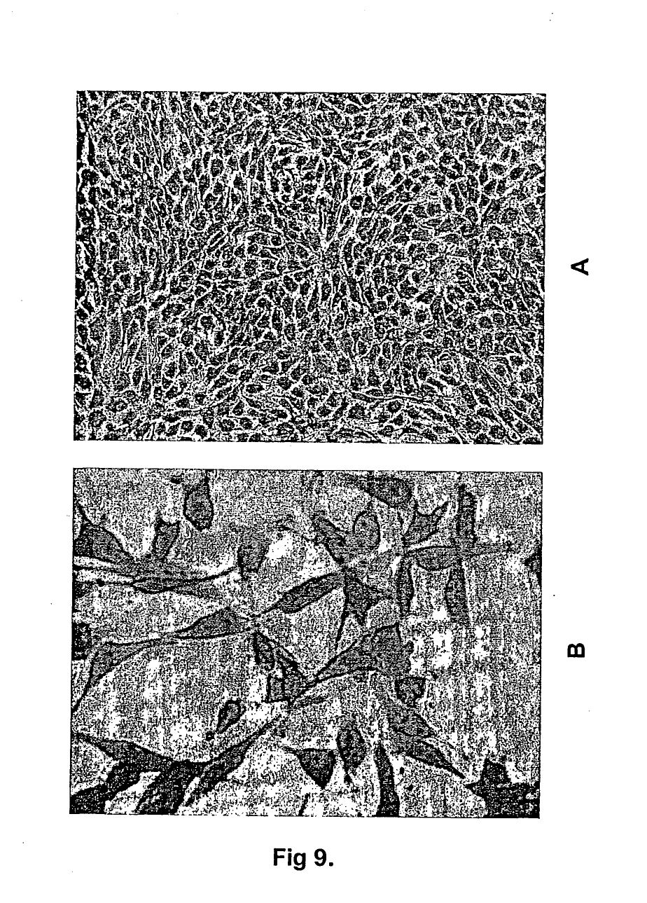

FIG. 10 relates to Example 7. FIG. 10a shows colony-forming units derived from patient # 21-30. Stained with Safranin O. FIG. 10b shows colony-forming units derived from one cartilage biopsy. Stained with Gentian Violet.

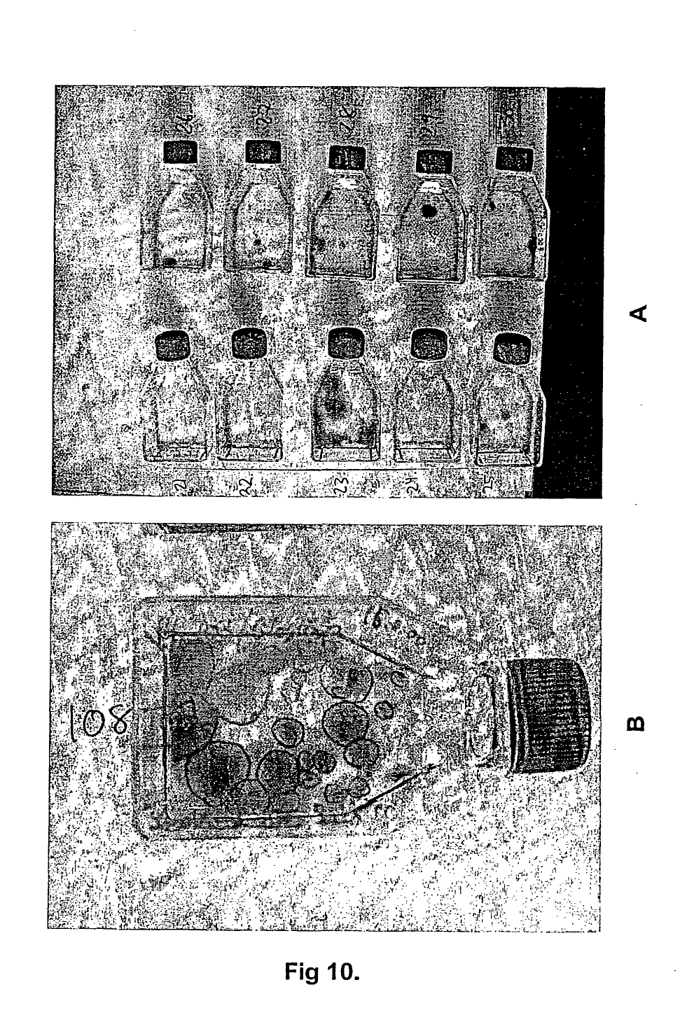

### DETAILED DESCRIPTION OF THE INVENTION

As appears from the above, the present invention is applicable to any cells obtainable from a mammalian tissue. In the following, the invention is illustrated mainly with a view on cartilage tissue but only for illustrative purposes. Thus, the invention should not be limited thereto.

The major role, in the success of cell implantation is profoundly dependent on the condition and homogenity of the cells to be implanted. In the case of chondrocyte implantation, it is of major importance that the chondrocyte culture is viable and inducible for proliferation and when implanted capable of providing a sufficient matrix production. It is important that the chondrocytes to be implanted are cultured under the most gentle and strictest culture methods avoiding unnecessary enzymatic damage of the cell membrane, and at the same time obtaining the most optimal chondrocyte culture to produce a healthy hyaline artricular cartilage. The use of a cell culturing method, which is very gentle to the chondrocytes is of utmost importance in order to obtain sufficiently healthy implant cells, capable of interacting with the surrounding cartilage in vivo. The same applies to other cells, which are obtained from mammalian tissue and cultured outside of a mammal and then transferred back to the same mammal (autologous) after cell culturing.

In a report by Brittberg et al., (1994, New Engl. J. Med.; Minas, T., Am. J. Orthop. 1998, 11:739) describing some of the first results of autologous chondrocyte implantation in patients with cartilage defects, it was described that two months after implantation of cultured chondrocytes, the dominant part of the repair tissue develops into fibrous cartilage not possessing the same biochemical properties as hyaline cartilage. This problem may be related to the observation that isolated chondrocytes cultured on plastic surfaces dedifferentiate into fibroblast like cells and not into the chondrocyte phenotype. This problem may also be true for other cells, which are cultured in tissue culture flasks until a monolayer is produced from the cells. In the case of a monolayer culture often the monolayer is released from the plastic surface by means of an enzyme like trypsin, and the cells released from the monolayer culture are transferred into another tissue culture flask for further culturing.

In the injured tissue site, a cell dependent differentiation process occurs after cell implantation. The cultured cells undergo a series of cellular transitions with a final differentiation to the original phenotype at the site of cell implantation. The repair of the tissue is thus dependent on the level and type of differentiation of the implanted cells. Less differentiated and immature cells, for instance isolated by the cartilage explant method according to the present invention, are cells capable of taking part in the local repair process because these cell types have not lost their potential to further differentiate and mature at the site of implantation. The final maturation of the implanted cells should be guided by the local environment at the site of implantation in order to secure a correct phenotypic matrix synthesis and cell development. In this way, the cells will produce repair tissue such as hyalin cartilage with the proper morphologic characteristics of the injured tissue. In comparison, cells, which are terminal differentiated such as mature chondrocytes present in chondrons, are less capable of taking part in the repair process after cell implantation. These cell types are often co-isolated by the conventional collagenase isolation method described elsewhere in this text.

The explant system builds on the concept of using the cartilage matrix as the “binding and delivery” system of growth and differentiation factors to the various cells present in the extracellular matrix. Growth and differentiation factors present in fetal calf serum or added separately to a medium as recombinant peptides are capable of binding to the cartilage via binding proteins such as, e.g., decorin, biglycan and fibromodulin, present in the matrix. Such a binding and delivery system might increase the half-life time of the various growth and differentiation factors present in the culture medium meaning that these and other factors are more protected from proteolysis when first bound to its natural intermediate matrix molecules—the “delivery vehicles” to the various cell receptors.

Additionally, a higher stimulation of cells by growth and differentiation factors, could be speculated, because of a higher local accumulation of the factors around the pericellular rim, in the territorial as well as in the interterritorial compartments, compared to the concentration of these factors present in the culture medium. A higher local concentration of these factors, bound to its natural ligand in the matrix and later “delivered” to the binding receptors (e.g. IGFI—R, TFG beta-R, bFGF-R) induce receptors to cluster formation on the plasma membrane and further signal transduction and growth stimulation.

For the stimulation and growth of the various cells in the matrix, it is a more natural and efficient process for the cells to start their growth and differentiation in their natural extracellular matrix, instead of stripping off the cells from its natural matrix components, the history of the cell, by the conventional collagenase digestion method.

The ability of the chondroblast/chondrocytes to remain viable in cartilage explants in the absence of fetal calf serum or other added growth factors further suggest, that the cells are protected by the extracellular matrix. Additionally, it has recently been shown in our laboratory that the conventional collagenase digestion of the matrix damages many isolated cells significantly and induce other to cell death via cell apoptosis. These results further emphasise the importance of maintaining as much as possible of the cells environment and matrix integrity before the cultured cells in the culture flask are released for final implantation.

A second problem in the culturing of mammalian cells is to obtain a homogeneous cell culture containing cells of the same origin and at the same time being in good condition. Proteolytic enzymes are commonly used to release cells from mammalian tissue and it destroys the normal structure of the tissue in such a manner that no integral part of the tissue is present after enzymatic treatment. However, the present inventors have observed that there is a huge variation in the quality and quantity of the released cells dependent on the sensitivity of the cells or matrix to the proteolytic enzymes employed and on the location and types of cells within the mammalian tissue.

Today, mammalian cartilage cells are released using approximately between 100 to 150 mg of proteolytic collagenase/mg of tissue material for a certain period of time. During this time period cells and other matrix components of the tissue are released. Undigested and disintegrated tissue material is removed from the released cells by for instance, centrifugation or filtration and the cells are cultured in a growth medium. However, the present inventors have found that the use of such an enzymatic method results in isolation of cells of different phenotypes and origin. During the further culturing, the most viable cells will proliferate and the final culture will be a mixture of cells. Moreover, it has been observed that such an initial enzymatic treatment of the cells may lead to unwanted properties of the cultured cells when implanted into a mammal.

In short, the above tissue culture system, in the text described with cartilage explants as one example of propagating cells in vitro from explants, has a number of other advantages over known chondrocyte isolating and culture methods. As earlier mentioned, cartilage derived cells in the explants maintain their differentiated state in the matrix while “natural” cell stimulation and cell propagation proceed. Second, the cells are not exposed to high concentration of damaging proteolytic activity used for instance by collagenase digestion of the matrix. Third, the culture system is easier to work with in the production unit and fourth, the production expenses are less when compared to conventional enzyme digestion methods, because the explant system needs less work and care during the cell isolation process as well as during the general cell culture process.

The object of the invention is to provide a general method in which a piece of mammalian tissue is collected from a mammal and in which the cells within the piece of mammalian tissue do not need to be released prior to culturing. The culturing of the piece of mammalian tissue results in one or more colony forming units, which only undergo a treatment with enzymes for final release of the colony forming units from the surface of the tissue culture flask and furthermore, the method is autologous.

The invention also related to a method for autologous treatment of a mammal suffering from a tissue or tissue related disorder, the method comprising administering to the mammal in need thereof cells obtained by the production method of the invention.

Examples of relevant tissue or tissue related disorders are those selected from the group consisting repair of hyalin articular cartilage defects in joints, repair of hyalin/fibrous cartilage defects of the intervertebral discs, repair of larynx defects related to hyalin/fibrous cartilage, remodelling of connective tissue containing elastic cartilage used in plastic surgery methods, repair of defects bone structures related to osteoarthritis, osteoarthrosis, osteoporosis, defect bone structures do to complicated fractures and atrophic pseudo arthrosis, repair of insufficient jaw bone structure for instance related to implantation of Titanium screw for tooth repair, treatment and repair of skin burns or other skin defects related to traumas and skin related tumors as for instance hemangiomas and malignant tumors such as melanomas, repair of the ventricular wall of the heart after infarction, repair of CNS related diseases such as Parkinson, Alzheimer, dementia, multiple sclerosis and other systemic brain diseases, repair of retinitis of different pathological origin, repair of pancreatic tissue related to diabetes type I and/or II, repair of liver cirrhosis, fatty liver and for the reconstruction of liver tissue after the removal of primary liver cancers as well as liver metastases and recreation of liver structures in children with birth defects.

Definitions

In the context of the present application and invention the following definitions apply:

The term “Autologous Cell Implantation/transplantation is intended to mean a process wherein a biopsy (or cells) is removed from the mammal and these cells are cultured and grown and later returned to the same mammal.

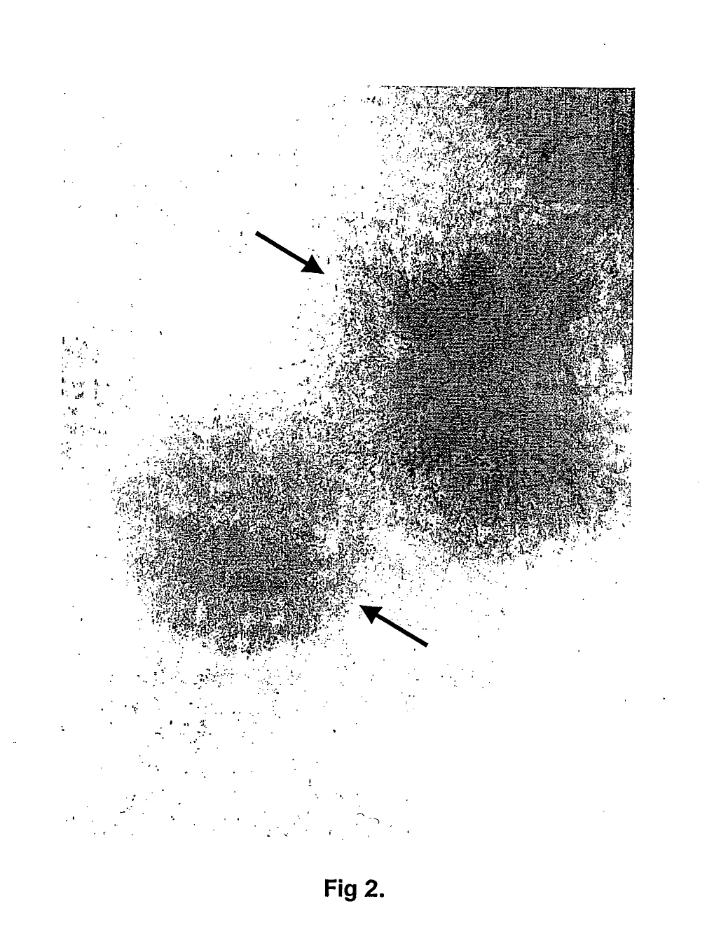

The term “cell colony forming unit” is intended to mean a colony of cells having the same origin, i.e. being derived from one cell such as, e.g., an immature cell which has migrated out from a piece of mammalian tissue explant and started to divide to produce a cell colony outside the piece of mammalian tissue explant (FIG. 2.). The cell colony-forming unit contains cells in several layers, the first one being adhered to the surface of the tissue culture flask in which the cell colony-forming unit is expanded and grown. The cell colony-forming unit increases in size both vertically, by increasing the diameter of the cell colony-forming unit, and horizontally. Thus, a cell colony-forming unit is typically different from a monolayer of cells since it is a multilayer of cells forming a clone of cells.

The term “mammalian tissue explant” is intended to mean a part of a mammalian tissue, which has been explanted from the mammal by use of a suitable instrument. An explant may contain more than one kind of tissue, e.g. in the case of explantation of tissue from the knee, the explant may contain cartilage tissue as well as bone tissue. In the present context the explantation of the tissue is suitably performed in a reproducible manner, i.e. by means of a well-defined instrument and a well-defined method.

The term “piece of mammalian tissue explant” is intended to mean a part of the mammalian tissue explant defined above. Advantageously, the part has a well-defined size and is obtained from the mammalian tissue explant by cutting or slicing the explant into smaller pieces. For some purposes it may be desired to use a specific part of the explant. Normally, the part is collected from a mammalian and used to produce one or more cell colony forming units as defined above. By aid of certain factors, the immature cells are able to migrate from the piece of mammalian tissue explant out into the growth medium. In the growth medium as well as in the explants the immature cells start to proliferate into several cells and thereby producing cell colony-forming units. One immature cell gives rise to one cell colony forming unit. However, several immature cells may migrate out from the piece of mammalian tissue explant and give rise to several cell colony-forming units within one and the same tissue culture flask. In the piece of mammalian tissue explant several kinds of cells may be present but in the present context it is of interest that the immature cells are capable of migrating out from the piece of mammalian tissue explant into the growth medium and start proliferating. In comparison, the mature cells remain in the piece of mammalian tissue explant and the piece of mammalian tissue explant functions in this way as a filter for withholding certain cells, and selecting other cell types. Thereby a cell selection is obtained by the aid of the piece of mammalian tissue explant.

The explant cell culturing system is built on the concept of using the cartilage matrix as the first binding and delivery system of growth factors and cytokines to the immature cells present in the extracellular matrix. The growth factors and cytokines present in the growth medium start to diffuse into the explants and after a fixed time, various concentration gradients of growth factors and cytokines are established in the matrix. As a result, it is speculated that the immature cells starts to migrate against the established gradients. After approximately 1-2 weeks of culturing are on the surface of the explants and finally establish cell colony-forming units outside the tissue.

The term “immature cell” is intended to mean a cell equal to a stem cell or a cell close to progenitor level such as, e.g., a prechondroblast or even a chondroblast. The immature cells are capable of migrating within the piece of mammalian tissue explant to the surrounding growth medium and the cells are also capable of migrating in the growth medium itself. In the example of the cartilage explant system, cell migration in the matrix will be limited to the periphery of the cartilage biopsy because the stimulated cells in the interior of the cartilage matrix will be trapped by the heavily composed collagen network. The migration may be influenced by several factors. After the cells have migrated out of the piece of mammalian tissue explant, the immature cells are capable of proliferating to form one or more cell colony-forming unit as defined above.

The term “migration” is intended to mean that the immature cells are mobile. The cells are capable of limited migration within the piece of mammalian tissue explant, from the piece of mammalian tissue explant into the growth medium and within the growth medium.

The term “producing” is intended to mean large-scale proliferation of the immature cells, into small or large cell colony-forming units. The produced cells originate from the same parent cell and will also differentiate into the same type of cells.

The term “matrix” is intended to mean extracellular proteins of tissue.

The term “rinsing” is intended to mean subjecting the piece of mammalian tissue explant to a fluid in such a manner that blood, unwanted material of mammalian tissue and/or other undesired components are removed. Such components may have been attached to the piece of mammalian tissue explant and they may be removed by using, e.g., an aqueous medium.

The term “aqueous medium” is intended to mean a medium, which contains water or a water-soluble liquid. Preferably the medium contains water. The content of water may be from 1-100% w/w, normally in a range of from about 60-100% w/w. An aqueous medium of interest is a physiological medium having a pH from about 5 to about 9. However, the aqueous medium should be designed in a way to minimise harm to the piece of mammalian tissue explant, while it on the other hand is capable of removing or reducing the amount of unwanted substances. The medium may furthermore comprise serum or other components in order to improve the rinsing effects. An example of a suitable medium is PBS or DMEM/F12.

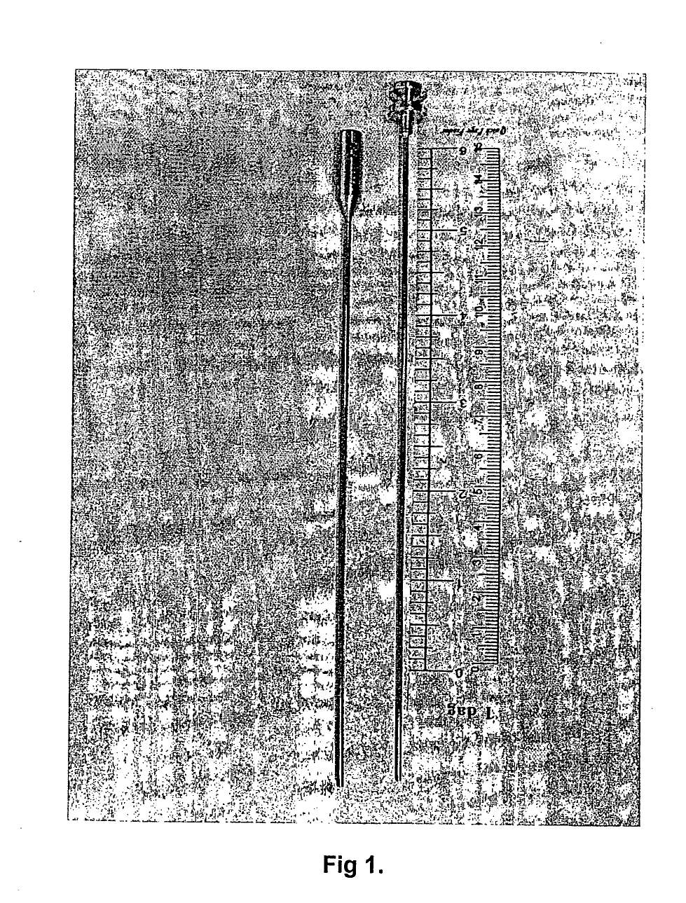

The term “instrument” is intended to mean an instrument having a sharp end portion for inserting the instrument into the tissue and a well-defined lumen for carrying an explant of the tissue. The instrument may be in the form of a needle such as e.g., shown in FIG. 1. The size of the instrument is not important and may vary depending on the amount of the piece of mammalian tissue explant necessary to obtain the cell culture.

The term “partial treatment” is intended to mean a treatment of the piece of mammalian tissue explant in order to obtain an explant having an opened up structure of the explant. The treatment is so gentle that the tissue is not disintegrated and it will not substantially degrade the tissue explant, i.e., no release of cells or other parts of the tissue explant occurs. The partial treatment facilitate diffusion and/or migration of growth factors and metabolites and immature cells into or out from the tissue explant.

The term “growth medium” is intended to mean a medium capable of inducing or providing migration of immature cells from a tissue material into the surrounding medium, attach to the surface of a tissue culture flask in which the piece of tissue explant and the growth medium is contained and further proliferation and differentiation of the immature cells into colony forming units.

The term “tissue culture flask” is intended to mean any conventional tissue culture flask well known for a person skilled in the art. The tissue culture flask may have different morphologies and sizes. However, the tissue culture flask must be able to permit cells to adhere and grow and comprise of at least one end, which can be opened and closed.

The term “cartilage” is intended to mean the connective tissue that contains stem cells, prechondroblasts, chondroblasts, chondrocytes embedded in the extracellular matrix. Cartilage includes hyalin, hyalin articular, elastic and fibro cartilage.

The term “release” is intended to mean release of the cells from the surface of a tissue culture flask to which the cells have adhered during growth and furthermore, it means releasing cells adhering to each other. The release of the cells enables the possibility to harvest the cells from the cell culture. An example of an agent to be used for the release of cells is trypsin or a chelating buffer system with EDTA. The release of the cell colony forming units may also be performed using cell scraping.

The term “electromagnetic induction” is intended to mean an electromagnetic induction, which results in signal transduction in e.g. the cells present in the piece of mammalian tissue material and thereby cells are activated in the piece of mammalian tissue explant. The electromagnetic induction may preferably be performed using an intermittent or a continuous pulse field from about 0.1 to about 0.8 mT. The only limitation of the size of the electromagnetic induction field is that the cells should remain viable and able to proliferate after treatment with electromagnetic induction.

The term “carrier” is intended to mean a liquid, a fluid, semi-solid or a solid carrier. For pharmaceutical purposes the carrier must be pharmaceutically acceptable which is a standard that is well known for a person skilled in the art. Thus, a pharmaceutically acceptable carrier is intended to denote any material, which is inert in the sense that it does not have any substantial therapeutic and/or prophylactic effect per se.

The term “biological material” is intended to mean any component of a cell, which may be produced by the cells of the cell colony-forming unit to an amount, which permits the production of the component in large scale. The component may be e.g. any protein of interest such as, e.g., enzymes, collagens, proteoglycans, glycosaminglycans and hyaluronic acid.

The term “biochemical analysis” is intended to mean any biochemical analysis, which may be performed on a piece of mammalian tissue explant. The analysis may be performed to evaluate the content of DNA, RNA and/or a certain protein or the activities of certain genes ongoing in the cells derived from the piece of mammalian tissue explant. Thereby the status of the mammalian tissue explant will be obtained. For example the status of a piece of cartilage obtained from a mammalian may indicate future cartilage defects, such as osteoarthritis, and thus giving an indication for the need of pre-treatments to prevent the development of such defects.

The term “tissue disorder” is intended to mean tissue disorders in tissues such as cartilage, bone, connective tissue, muscle tissue, skin tissue, mucosal tissue, brain tissue, heart tissue, kidney tissue, pancreas tissue and liver tissue defects in mammals. Cartilage:—for hyalin articular cartilage defects in joints and for the repair of hyalin/fibrous cartilage defects of the intervertebral discs. Further, for larynx defects related to hyalin/fibrous cartilage connective tissue disorders. Bone:—defects bone structures related to osteoarthritis, osteoarthrosis, osteoporosis and defect bone structures due to complicated fractures and atrophic pseudo arthrosis. Further, insufficient jaw bone structure. Connective tissue disorders:—related to skin burns or other skin defects related to traumas and skin related tumors as for instance hemangiomas and malignant tumors such as melanomas. Muscle:—disorders of the ventricular wall of the heart after infarction. Skin:—skin burns or other skin defects related to traumas and skin related tumors as for instance hemangiomas and malignant tumors such as melanomas. Brain:—for CNS related diseases such as Parkinson, Alzheimer, dementia, multiple sclerosis and other systemic brain diseases. Eye:—retinitis of different pathological origin. Heart:—infarction of the ventricular wall of the heart. Pancreas: pancreatic tissue related to diabetes type 1 and type 2. Liver:—for the repair of liver cirrhosis, fatty liver and for the reconstruction of liver tissue defects after the removal of primary liver cancers as well as liver metastases. Furthermore, disorders of the liver structures in children with birth defects.

Method of Producing Cell Colony-Forming Units in vitro in a Production Plant

In one aspect the invention relates to a method of producing cell colony-forming units in vitro, using the steps of (a) growing a piece of mammalian tissue explant in a growth medium to obtain cell colony forming units from immature cells from the piece of mammalian tissue explant and (b) harvesting one or more cell colony-forming units for use in autologous cell implantation/transplantation methods.

By using an intact piece (i.e. a piece which has not been subject to enzymatic treatment resulting in release of cells) of mammalian tissue explant the immature cells are kept within the matrix structure during the initial part of the culturing procedure. This preserves the intermediate environment of the cell with its macromolecules, including cell binding proteins, as well as chondron organization of the fibrillar network in the territorial matrix around the individual cells resulting in a controlled cell growth in a phenotypically stable fashion.

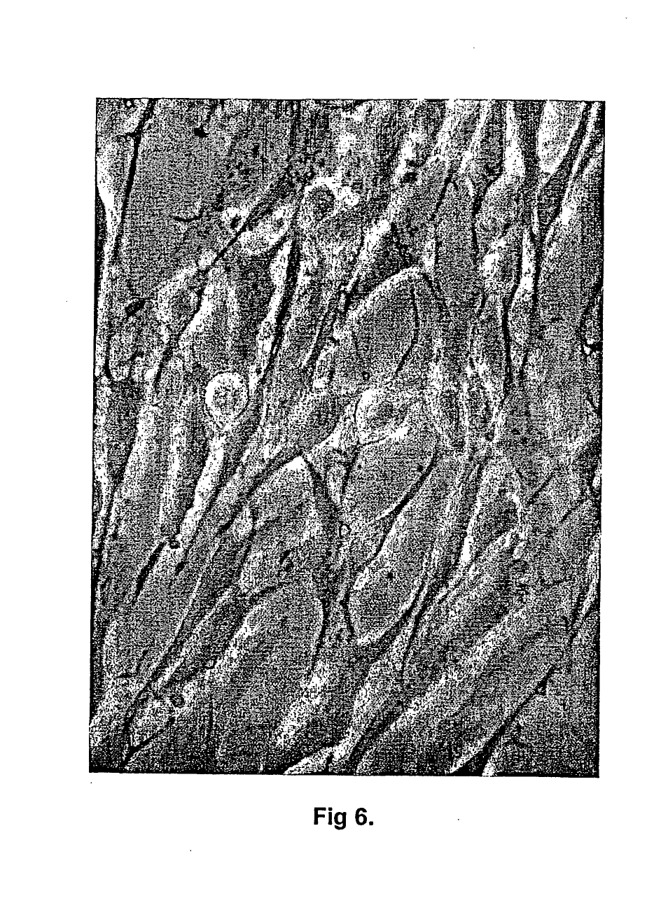

The method may comprise a step of migration of the immature cells from the mammalian tissue explant into the growth medium. The migrated cells are cultured in a three dimensional system as small and large colonies of cells on plastic surfaces. The colony forming units produced from the migrated cells turns out to be very confluent after a certain period of growth. However, during the production period the colony-forming units are not released from the plastic surface by the aid of a release medium. Accordingly, damages of the cells are avoided and further cell differentiation is maintained through the entire culturing process. It has previously been found that cells; like chondrocytes, when they are placed in low density monolayer culture and spread along the surface of the tissue culture flask, the monolayer cells cultured resemble fibroblasts and do not appear to be chondrocytes (see FIG. 6). The colony-forming units are finally released from the tissue culture flask before the time of autologous cell implantation.

Mammalian Tissue Explant and Handling Thereof

The mammalian tissue explant used in a method according to the invention is selected from tissue originating from the cells selected from the group consisting of mesenchymal cells, ectodermal cells and endodermal cells, preferably the group consisting of cartilage; bone such as, e.g., bone marrow, connective tissue; muscle tissue such as, e.g., smooth muscle tissue, heart tissue, liver tissue and skeletal muscle tissue; skin tissue such as, e.g., periosteum; mucosal tissue; brain tissue; kidney tissue, pancreas tissue and pancreatic islets, preferably cartilage, such as elastic, fibro, hyalin or articular hyalin cartilage.

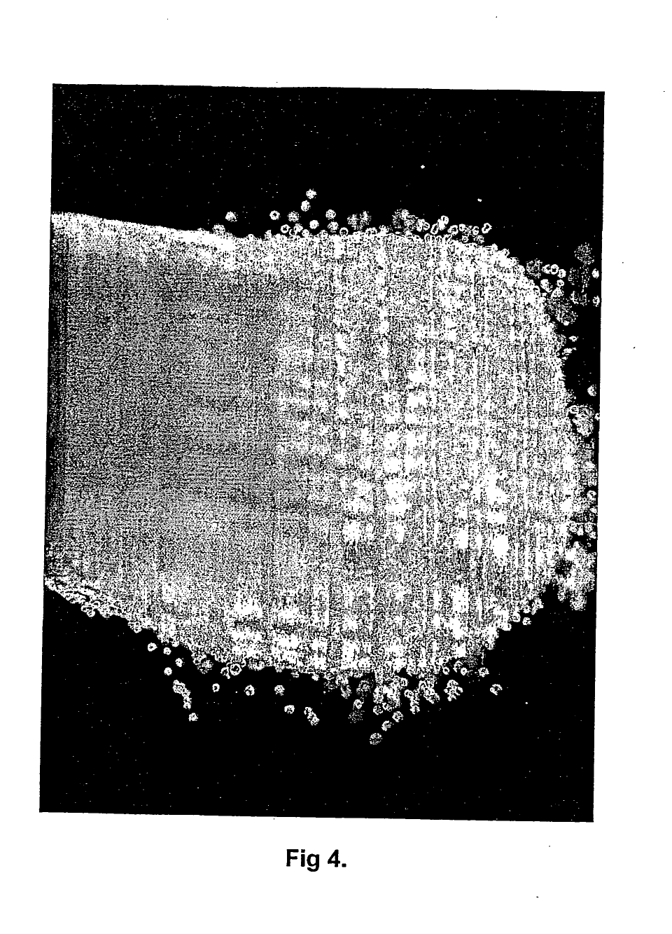

The mammalian tissue explant may be obtained by an instrument as defined above. The instrument is designed for optimally obtaining a piece of mammalian tissue without causing much harm to the surrounding tissue. The well-defined lumen of the instrument may be of variable size and form dependent on which tissue to be harvested. Thus, the cross section may be in a circular or polygonal form and the diameter of the circle or the polygon may be in a range of from about 0.3 to about 100 mm such as, e.g., from about 2 to about 10 mm. Suitably, the overall shape is cylindrical. The length of the instrument is not important provided that it has a length, which makes it suitable for withdrawal of the specific mammalian tissue explant and for handling of the instrument. One end of the cylindrical instrument is sharpened in order to facilitate the penetration of the specific tissue and it has a conical shape. The instrument may be made of any suitable material provided that it has a suitable strength to enter and penetrate the tissue of interest. With respect to cartilage the instrument should be made of a relatively enforced material such as, e.g., metal or steel. The explant may be released from the instrument in many ways, preferably cut or stamped out, more preferably stamped out. An example of an instrument suitable for use in the withdrawal of mammalian tissue explant is shown in FIG. 1 (see also FIG. 4 where the instrument has been used to obtain a piece of mammalian tissue explant, such as a piece of cartilage). The piece of cartilage may be obtained from a joint such as, e.g., a knee of a mammal, such as human, domestic and/or racing animal including horses and camels. In that particular case the instrument is designed as a needle of enforced material with the ability of penetrating the cartilage and obtaining an explant. The needle may have different diameters of the sharpened end for the possibility to obtain different sizes of the explant. The instrument may be disposable or used several times.

The mammalian tissue explant may be stored prior to step (a). The mammalian tissue explant may be stored as either the entire or at least part of the original mammalian tissue explant. The storage may be at a temperature from about −180° C. to about 37° C., such as from about preferably from about −180° C. to about −70° C. or from about −70° C. to about 10° C., preferably at a temperature of about −180° C., about −70° C., about 4° C. or about 8° C., more preferably about −70° C. and even more preferably at about −180° C. The storage may be in a cold room, conventional freezer, a laboratory freezer at the temperature from about −70° C. to about −80° C., in liquid nitrogen or as a lyophilised tissue optionally together with a suitable solid carrier. Prior to step (a) the stored piece of mammalian tissue explant will be removed from the storage place and in certain cases the temperature is gradually increased from the storage temperature to a higher temperature prior to step (a) in the method.

Treatment of the Mammalian Tissue Explant Before Culturing

Normally, the method of producing cell colony forming units further comprises additional steps such as a step of rinsing the piece of mammalian tissue explant prior to step (a). The rinsing is performed to remove undesired components adhered to the mammalian tissue explant, which might inhibit and/or affect the migration and proliferation of the immature cells and thereby inhibit the formation of cell forming units. The rinsing may be performed by an aqueous medium having a pH from about 5 to about 9, preferably close to pH 7.4. Examples of aqueous mediums are any form of physiological salt solutions or PBS as described in Example 1.

Other additional steps may include treatments performed prior to step (a), such as partial treatment using one or more proteolytic enzymes alone or in combination with pretreatment with an aqueous medium. Partial treatment of the piece of mammalian tissue explant with one or more proteolytic enzymes is performed under such conditions which enables an opening up of the structure of the mammalian tissue explant and thereby facilitating the diffusion of growth factors and migration of immature cells in and/or out from the mammalian tissue explant prior to step (a). Hereby the piece of mammalian tissue explant remains intact in such as way that no cells or other parts are released from the piece of mammalian tissue explant prior to culturing. The partial treatment of the piece of mammalian tissue may be performed utilising one or more proteolytic enzymes in a concentration ranging from about 1 to about 90 U/mg of the mammalian tissue explant such as, e.g., from about 1-10 U/mg, from about 1-5 U/mg or about 2.5 U/mg. The partial treatment of the piece of mammalian tissue explant may be performed utilising proteolytic enzymes such as proteinases and/or trypsin, preferably proteinases selected from the group consisting of aspartate proteinases like Cathepsin D, cysteine proteinases like Cathepsin B, L, S, K and Calpains I and II, serine proteases like neutrophil elastase, Cathepsin G and Proteinase 3 and metallo proteinases like MMP-1, MMP-2, MMP-3, MMP-4, MMP-5, MMP-6, MMP-7, MMP-8, MMP-9, MMP-10, MMP-11, MMP-12, MMP-13, MMP-14, MMP-15, MMP-16, MMP-17, MMP-18, MMP-19, MMP-20 and/or trypsin. Pre-treatment of the piece of mammalian tissue explant with an aqueous medium may be performed with a aqueous medium having a pH which is at least ±0.5 from the pH of 7.4 (physiologic pH), such as a pH within the range of from about 4 to about 6.9 such as from about 5 to about 6.9, from about 6 to about 6.9 such as, e.g., about pH 6.5, or a pH within the range of from about 7.9 to about 10 such as, e.g., from about 7.9 to about 9, from about 7.9 to about 8.5 such as, e.g. a pH about 8.0.

Culturing of the Piece of Mammalian Tissue Explant

The piece of mammalian tissue explant is optionally retained during the course of culturing such as, e.g., until one or more of cell forming units are harvested. By leaving the tissue during the entire process of culturing the immature cells producing the cell forming units are less stressed during the method and, thereby, an improved result is expected.

Furthermore by avoiding a step of removing the piece of mammalian tissue explant, the tissue culture are less exposed to surrounding microbial agents which might enter into the tissue culture flask during a step when it is necessary to open the tissue culture flask to insert or remove components.

Growth Medium

The piece of mammalian tissue explant is cultured in a tissue culture flask using a suitable growth medium or, alternatively, two or more suitable growth media. One growth medium may be used throughout the method according to the invention or, alternatively, two or more of different growth media may be used during the method of culturing. The growth medium (media) may include different components when two or more different growth media are used during the method of culturing. The growth medium (media) comprise(s) one or more components selected from the group consisting of metabolites such as, e.g., carbohydrates, lipids and amino acids; vitamins; growth factors; cytokines; minerals and antimicrobials, preferably the growth medium comprises mammalian serum, such as serum from a human, a domestic and/or racing animal (e.g. a horse or a camel), preferably the mammalian serum is autologous to the mammalian tissue explant used throughout the above mentioned method. Example of a suitable growth medium is DMEM/F12 with 10-20% w/w fetal calf serum.

According to one embodiment of the invention the first growth medium comprises at least a non-autologous serum, which is used initially in the method. Before harvesting and release of the cells, the first growth medium is changed to a second growth medium comprising at least an autologous serum to enable reduction and/or removal of undesired immunogenic components from the non-autologous serum used in the first growth medium.

Alternatively, the growth medium may be based on a synthetic or a semi-synthetic medium, i.e. a medium without or substantially without mammalian biological material. To such a medium other factors like the ones mentioned above may be added. In U.S. Pat. No. 6,150,163 is given an example of a suitable growth medium.

If necessary, the method of the invention also includes a step of enrichment of the growth medium with growth factors into the mammalian tissue explant and/or to the growth medium in the tissue culture flask.

Additionally, a method according to the present invention above may employ continuous and/or pulsed delivery of growth medium, components of the growth medium or other agents to the tissue culture flask, thereby a controlled delivery of the above-mentioned components or agents is obtained. In such a case the tissue culture flask is provided with an inlet and an outlet end portion (openings) for the continuous and/or pulsed delivery. In some cases it may be advantageous that the continuous and/or pulsed delivery is established in such a manner that a difference in pressure is obtained between the inlet and the outlet ends.

The method according to the invention is performed in a conventional tissue culture flask as defined above. However, the size or form of the tissue culture flask is of no importance provided that it is suitable for the purpose and may be adapted to the amount of cell colony forming units produced using the above mentioned method.

Release of the Cell Colony-Forming Units

The cell colony-forming units may be released by use of a release medium. The step comprising contacting the cell colony-forming units with a release medium, which enables release of the cells of the cell colony-forming units from the tissue culture flask and from each other, preferably the release medium is an aqueous medium, which optionally comprises one or more enzymes selected from trypsin and other enzymes. Alternatively the release medium is a buffer such as a PBS buffer without divalent metal ions. The release of the cell colony-forming units are performed to remove the cell colony-forming units from the tissue culture flask and/or for the ability to transfer the cell colony-forming units into new tissue culture flask to enable further growth of the cell colony-forming units.

Alternatively a step of subjecting the piece of mammalian tissue explant to electromagnetic induction may be utilised in combination with the production method. The electromagnetic induction step may be performed to facilitate proliferation and migration of the immature cells from the piece of mammalian tissue explant.

In certain circumstances the cell colony-forming units may be transferred to another tissue culture flask for further culturing.

Cell Colony-Forming Units (CFU)

The cell colony-forming units produced by the method are derived from immature cells derived from the three tissue layers such as mesemchymal, ectodermal and endodermal layers, preferably mesemchymal cells derived from stem cells, prechondroblasts, chondroblasts, chondrocytes, preosteoblasts, osteoblasts, osteocytes, premyoblasts, myoblasts, myocytes, cementoblasts, cementocytes, odontoblasts, odontocytes, ameloblasts, amelocytes, fibroblasts or fibrocytes.

During the culturing of the piece of mammalian tissue explant an analysis may be performed as soon as the immature cells have started to proliferate into cell colony-forming units for the ability to verify the identity of the cells within the cell colony-forming unit. Preferably the piece of mammalian tissue explant is cartilage, such as elastic, fibro, hyaline or articular hyalin and the immature cells in the explant proliferate into prechondroblasts or chondroblasts or chondrocytes. If the piece of mammalian tissue explant is bone, the immature cells multiply into preosteoblasts or osteoblasts or osteocytes. The condrocytes or chondroblasts may be analysed by morphogenic analysis in light microscopy or detection of collagen type II or hyalin synthesis and secretion by methods involving RNA or protein analysis such as PCR or Western Blot analysis.

The method according to the invention is normally performed at a temperature of about 37° C.±10° C. However, as shown in the Examples a lower temperature may be used in those cases where a slower growth is wanted.

It should be mentioned that a method for culturing cartilage cells is subject of a patent application PCT/EP00/07111 filed by the same inventors. If relevant and only for identical matter a disclaimer may be introduced.

Composition of One or More Cell Colony Forming Units

The invention further relates to a cell colony-forming unit or a group of cell colony forming units or suspensions thereof produced according to the method of the invention described under “Method of producing cell colony forming units in vitro”.

Additionally the invention relates to a composition comprising one or more cell colony forming units produced according to the invention, and a carrier. Preferably one or more cell colony forming units and at least part of the carrier are autologous such as autologous serum. The carrier is defined above under “definitions”.

The cells or one or more cell colony forming units are normally present in the carrier in suspended or dispersed form.

The carrier may include pH-adjusting agents, solubilizing agents, wetting agents and buffering agents. The carrier may also include additives like e.g., suitable salts such as salts with alkali metals or alkali earth metals, such as sodium, potassium, calcium and magnesium, as well as e.g. zinc salts. Other examples are stabilisers, preservation agents, osmotic or isotonic adjusting agents, non-ionic detergents, antioxidants as well as serum, such as autologous serum. The carrier may also include metabolites such as, e.g. amino acids, lipids and/or carbohydrates, nutrients and/or minerals and it may also include a therapeutically and/or prophylactically active substance such as a drug substance or an immuno suppressive agent.

Examples of semi-solid carriers are e.g. polyethylene glycols, glycofurols and a like.

The above-mentioned composition may be used as a pharmaceutical or a diagnostic composition or for isolation, purification or production of biological materials. The pharmaceutical composition can be used alone or with a carrier as described above. At least part of the carrier is preferably autologous, e.g. serum obtained from the same mammal as the mammalian tissue being used in the method of the invention The pharmaceutical composition may further contain components derived from matrix including collagen proteins such as, e.g., collagen types II, IV, IX and XI, proteoglycans such as, e.g., aggregans, decorin, fibromodulin and biglycan, and non-collageneous proteins such as cryoprecipitate, fibronectin, vitronectin, fibronogen, fibrillin, kistrin, echistatin, von Willebrand factor, tenascin and anchorin CII, and including other stimulation factors described in the patent application PCT/EP00/07111.

A pharmaceutical composition of the invention may be used in treatments of mammals suffering from tissue disorders, such as for instance cartilage and/or bone disorders. The mammals include humans or domestic or racing animals, including horses and camels.

A diagnostic composition of the invention may be used in diagnostic methods for the investigation of questions related to tissue disorders, such as the diagnostic method mentioned hereinafter.

Isolation, purification or production of biological materials may be any material such as DNA, RNA or protein obtainable from one or more cell forming units, such as those cell colony-forming units produced by the method of the invention.

Additionally the above-mentioned cell colony-forming units, the group of cell colony-forming units or the compositions may be used in medicine e.g. for the treatment of tissue disorders, such as cartilage and/or bone disorders.

Furthermore, the above mentioned cell colony-forming units, the group of cell colony-forming units may be used for the manufacture of a pharmaceutical composition for the treatment of tissue disorders, preferably treatment of cartilage and/or bone disorders in mammals, more preferably treatment of cartilage and/or bone disorders in humans or in domestic or racing animals including horses and camels.

Transportation Kit for Collecting Mammalian Tissue Explant of a Mammal

Furthermore, the invention relates to a transportation kit. A transportation kit to be used for collecting a mammalian tissue explant of a mammal, the kit comprising an instrument as described herein for collecting a mammalian tissue explant from a mammalian tissue and a transport container for preserving the mammalian tissue explant and, optionally, instructions for the use of the instrument as defined above.

The instrument is designed for optimally obtaining a piece of mammalian tissue without to much harm to the surrounding tissue. The well-defined lumen of the instrument may be of variable size and form depending on which tissue to be harvested. The explant may be released from the mammalian tissue in any way, preferably cut or stamped out, more preferably stamped out. An example of a suitable instrument is given in FIG. 1 in which a piece of cartilage is withdrawn. The piece of cartilage may be obtained from a knee of a mammal, such as human, domestic and/or racing animal including horses and camels. In that particular case the instrument is designed as a needle of hard material for the ability to penetrate the cartilage and obtain an explant. The needle may have different diameters of the end intended for penetration of the tissue in order to makes it possible to obtain different sizes of the explant. The instrument may be disposable or for use several times.

The instructions for use of the instrument are optionally included for the reproducibility, e.g., to obtain the same amount of mammalian tissue explant from the same position on the mammalian tissue.

The transportation kit may further contain a blood sample tube for collecting an autologous blood sample from the mammal, thereby enabling the possibility to use a growth medium comprising of autologous serum either throughout the method or the final part of the method as described above under “Method of producing cell colony forming units in vitro”.

The transportation kit may also be used for collecting a mammalian explant for use in a biochemical assay for the determination of DNA, RNA and/or protein.

Delivery Kit Comprising One or More Cell Colony Forming Units for Autologous Cell Implantation

The invention further relates to a delivery kit comprising at least a first and a second container, the first container comprising cells obtained by the method of the invention and a carrier, and the second container comprising a cartilage and/or an interface membrane. An example of useful cartilage and/or interface membranes is described in the patent application PCT/EP00/07111.

The delivery kit is suitable for use in the treatment of a mammal suffering from tissue disorders as defined above, like cartilage and/or bone disorders. The mammals are humans or domestic and/or racing animals induding horses and camels.

A Production Plant for the Production of One or More Cell Colony Forming Units Intended for Autologous Cell Implantation Methods

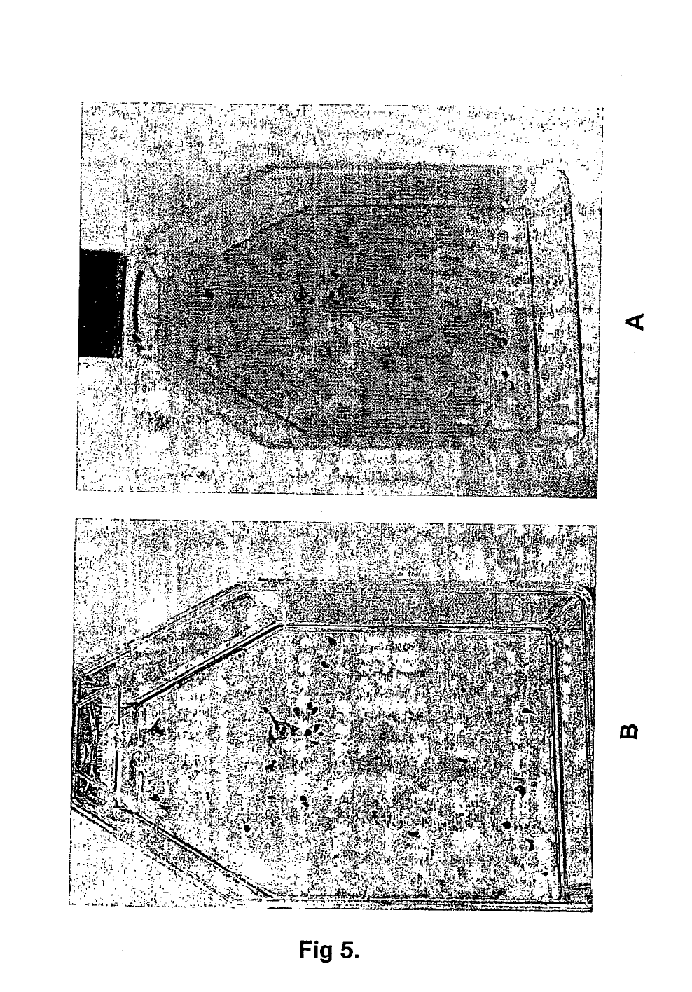

A production plant for the production of cell colony forming units in vitro from a mammalian tissue explant, comprising means for, application of a piece of the mammalian tissue explant to a tissue culture flask; application of a growth medium to the tissue culture flask; growing cells migrating from the piece of mammalian tissue explant into the growth medium, and application of a release medium to the tissue culture flask, as described an defined above. The production plant may furthermore comprise means for continuous or pulsed delivery of growth medium, release medium and/or one or more factors selected from the group consisting of metabolites such as, e.g., carbohydrates, lipids and amino acids; vitamins; growth factors; cytokines; minerals and antimicrobials. An example of a production plant intended for Autologous Cell Implantation/Transplantation methods is described in Example 1 and in the production scheme in FIG. 5.

Pharmaceutical Use and Formulations

In one aspect the cells or composition according to the invention is used for the manufacture of a pharmaceutical composition for treatment of diseases, in particular diseases as tissue disorders related to cartilage, bone, connective tissue, muscle tissue, skin tissue, mucosal tissue, brain tissue, heart tissue, kidney tissue, pancreas tissue and liver tissue defects in mammals and further defined under definitions, preferably cartilage and/or bone defects

In another aspect, the cells or composition according to the invention is used in a method for treating a mammal suffering from a tissue or tissue related disease, the method comprising administering to the mammal in need thereof a sufficient amount of a cell colony forming unit or a group of cell colony forming units or a composition.

In still another aspect, the cells or a composition according to the invention is used in an autologous method, for treating a mammal suffering from a tissue or tissue related disease, the method comprising administering to the mammal in need thereof a sufficient amount of a cell colony forming unit or a group of cell colony forming units or a composition. The method which, whenever relevant, uses a step in the production of the cell colony forming unit or group of cell colony forming units or composition of means of autologous material from the same mammal, such as autologous serum.

Diagnostic Method for Determination of Biological Activities

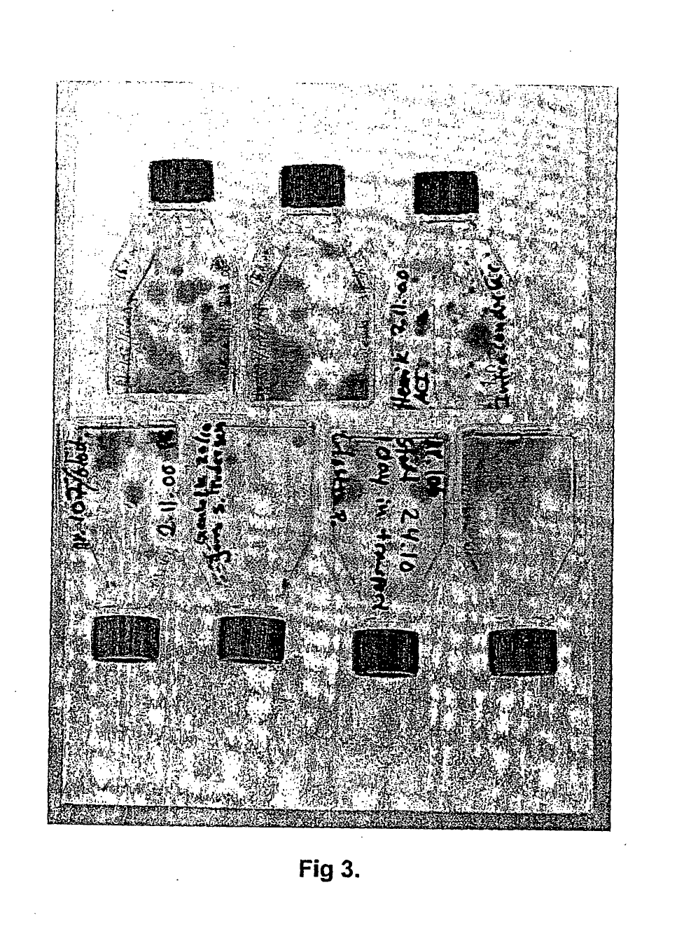

Additionally, the invention relates to a diagnostic method for the determination of the biological activities in a piece of mammalian tissue explant from an in vitro culture, the method comprising the steps of growing a piece of mammalian tissue explant comprising matrix and immature cells in a growth medium in a tissue culture flask, and subjecting at least a part of the content of the tissue culture flask to an investigation to receive information of the nature of the mammalian tissue explant and/or the immature cells, such as the possibility of a cartilage explant to produce cell colony forming units (FIG. 3). The diagnostic method may be an investigation such as a histochemically and/or a cytopathologically investigation. Furthermore, the diagnostic method may be an investigation like a biochemical analysis for the determination of DNA, RNA and/or protein.

Materials and Methods

Growth medium or sterile growth medium: DMEM/F12 containing 20% fetal calf serum. DMEM/F12 (cat no 31331-028) obtained from Life Technologies Inc., Rockville, Md., USA and fetal calf serum from Life Technologies Inc., Rockville, Md., USA.

The following materials are obtained from Life Technologies Inc, Rockville, Md., USA.

Trypsin 0.25%: cat no 25200-056

Fetal Calf serum: cat no: 10084-168, batch no 302528 1A

Fungizone: cat no 15290-026

Gentamycin: cat no 15710-031

Tissue culture flask: Easy flask: 25 cm², cat.no. 156367A

Tissue culture flask: Easy flask: 75 cm², cat.no. 156499A

PBS without Mg, Ca: cat no 14190-094

PBS with 1 mM of Mg and 1 mM of Ca

L-Ascorbic Acid 2-phosphate obtained from Sigma, cat no A8960

### EXAMPLES

### Example 1

Protocol for Culturing Cartilage Explants and Producing Cell Colony Forming Unit(s) in vitro for Autologous Chondrocyte Implantation (ACI) Use

The cartilage explant system is currently been used in the company's cell production unit related to the clinical trial: IBO-ACI-02, investigating the repair efficiency of cultured autologous chondrocytes implanted into articular cartilage defects in the knee. Until December 2001, the cartilage biopsies have been obtained from seven patients suffering from knee problems. The seven biopsy explants have each been cultured to about 11 million cells and implanted into the patients successfully at two major Danish hospitals in the Copenhagen area. The first outcome of the clinical investigations will be present during 2003/4.

In short, a cartilage biopsy was harvested from the patient's knee and immediately transferred into sterile growth medium, supplemented with L-ascorbic acid 2-phosphate [50 μg/ml (300 μmol/l)] and gentamicin sulfate [50 μg/ml (10 mmol/l)], Fungizone [2 μg/ml (2.2 μmol/l) in tissue culture flasks. The cartilage biopsy was then washed carefully with PBS with magnesium and calcium. When initially placed in the culture, it takes the cartilage explants several days depending on the donor material, to reach a constant metabolic state. Having reached such a steady state (steady state is a balance between synthesis and catabolism), the immature cells, prechondroblasts/chondroblasts were stimulated by growth factors present in the growth medium, which diffused through the cartilage matrix and bound to various binding proteins present in the matrix as well as binding directly to the selective cell receptors.

The stimulated (and proliferating) cells remain in the explants usually for one to two weeks (depending on the donor material) and then “leave” the explants, via cell migration and chemotaxis, into the culture medium for further attachment to the tissue culture flask to produce cell colony forming units.

After 3-5 weeks of culturing, smaller and larger cell colony-forming units had developed (see FIGS. 5A and 5B) and cells were harvested with trypsin.

Autologous growth medium containing 10% mammal serum instead of 20% fetal calf serum was added to the tissue culture flask (after rinsing) for further 2-3 days culturing before the cells were harvested and delivered as a cell suspension in a carrier to the clinic or a hospital.

### Example 2

Differential Isolation of Chondrogenic Cell Lines in the Cartilage Explant System

A cartilage biopsy was harvested from the patient's knee with a needle or a scalpel and transferred into sterile growth medium as described above. The cartilage piece was cut horizontal (tangential to the cartilage surface) with a sharp sterile scalpel into various zones. The cutting process was done under a microscopy in the laminar airflow hood in order to secure the proper separation and selection. The cartilage piece can be cut horizontal several times into various zones or the cartilage biopsy can be cut just one time into two layers. After separating the various cartilage layers into sterile tubes by cutting several times, the cartilage pieces were finally washed several times with PBS buffer with magnesium and calcium ions before adding the cartilage pieces to the tissue culture flasks. The further cell culturing process took place in a similar fashion as described in the protocol in Example 1.

### Example 3

Partial Enzymatic Treatment of Cartilage Explants With Crude Collagenase to Obtain CFU According to the Invention

A cartilage biopsy in the weighting of 100 mg was harvested from the patient's knee and transferred into sterile growth medium with antibiotic and fungizone as described in Example 1. The cartilage biopsy was washed in PBS with magnesium and calcium buffer and dissected vertical and horizontal with a sharp sterile scalpel into 24 mm cartilage pieces (explants). The cartilage explants in 25 ml growth medium were added to a 75 cm² tissue culture flask together with 10 U/ml (10 U/ml×25 ml=250 Units) crude collagenase from Clostridium Histolyticum (type 1A (C9891) or type 1 (C0130), or type VIII (C2139), type II (C6885) or type IV (C5138) or type V (C9283) (all obtained from Sigma chemicals). The tissue culture flask was then incubated at 37° C. with 5% CO₂ with low shaking for 18 hours.

After 18 hours of incubation with crude collagenase, the cartilage explants were washed in a 40 um cell strainer with 100 ml of PBS without Magnesium and Calcium and with 50 ml of growth medium in the same cell strainer. The various wash procedures of the explants are important in order to remove the collagenases present in the cartilage explants.

After the final wash procedure, the treated (predigested) cartilage explants (and possible released cells) were now added to a new 75 cm² tissue culture flask with new growth medium. The last part of the cell culturing process of the cartilage explants now took place as described for the protocol in Example 1.

### Example 4

Treatment of Cartilage Explants With Growth Medium Adjusted to pH 6.5

A cartilage biopsy in the weight of approximately 100 mg was harvested from the patient's femoral condyle and transferred into sterile growth medium with antibiotic and fungizone as described in Example 1. The cartilage biopsy was washed in PBS buffer with magnesium and calcium adjusted to pH 6.5 and dissected vertical and horizontal with a sharp sterile scalpel into 24 mm cartilage pieces (explants). The cartilage explants were added to a tissue culture flask with 25 ml growth medium adjusted (with sterile HCl) to pH 6.5 and incubated at 37° C. with low shaking for 18 hours. After incubation with “pH 6.5 growth medium”, the cartilage explants were washed in a cell strainer with 50 ml of normal physiologic PBS buffer with magnesium and calcium (pH 7.4) and with 50 ml of growth medium as used in Example 1. The washed cartilage explants were then added to a new 75 cm² tissue culture flask with new growth medium (25 ml) and cultured further as described in Example 1.

The CFU obtained were in accordance with the invention.

### Example 5

Postponing Cell Migration and Proliferation of Chondrogenic Cells in Cartilage Explant by Changing Incubation Temperature and Serum Concentration in the Growth Medium

A cartilage biopsy was harvested from the patient's femoral condyle and transferred into a sterile medium as described in Example 1. The cartilage biopsy was washed carefully with new growth medium as described in Example 1. The growth medium was at this point composed of 5% FCS giving a lower concentration of e.g., growth factors in the medium. The cartilage biopsy (100 mg) was cut horizontal and vertical with a sharp scalpel and the explants were added to the 5% growth medium (25 ml) before incubation of the culture flask (75 cm²) with explants at 37° C./5% CO₂ for 72 hours with temporary shaking of the culture flask (in a angel of 5 degree). After storing the explants in a resting state for 72 hours in the CO₂-incubator, the explants of cartilage can be transferred to a new tissue culture flask with new growth medium with 20% FCS and incubated at 37° C. with 5% CO₂ as described in Example 1. The further cell culturing process of the explants follows now the same protocol as described in Example 1.

The CFU obtained were in accordance with the invention.

### Example 6

Culturing of Periosteum Explants Harvested from Proximal Media Tibia

A flap of periosteum was harvested with an “elevator” from the proximal medial tibia and added to PBS buffer with magnesium and calcium in a sterile tube. The flap was cut perpendicular through the flap with a sharp scalpel into several pieces of tissue. The small pieces of periosteum tissue were washed another time in PBS buffer before adding the periosteum explants to growth medium as described in the protocol in Example 1.

When initially placed and stimulated by growth factors in the culture medium, it takes the periosteum derived immature mesenchymal cells 1-2 weeks to migrate out of the explant and to settle down on the plastic surface for establishing cell colony forming units. The mesenchymal progenitors with multi potential properties establish cell colony-forming units with fibroblastic appearance on the plastic surfaces. The colonies varied in sizes (diameter) and cell numbers and showed the same phenotypic morphologies as described for bone marrow stromal precursor cells.

The individual colonies could be further propagated into large cell numbers and used for autologous cell implantation methods and repair processes.

The knowledge of cell migration toward chemotactic factors, which takes place for a selected proportion of the mesenchymal, ectodermal and endodermal derived cells in explants, cultured in growth medium, was again applied and utilized in the isolation and selection procedure of immature mesenchymal cell—in this example immature mesenchymal cells present in the periosteum flap (the cambium layer) harvested from tibia.

The CFU obtained were in accordance with the invention.

### Example 7

Use of the Cartilage Explant System for Cartilage Analytical Purposes

Project title: “The viability study related to culturing of human chondrocytes obtained from patients undergoing knee arthroscopy for optimising Autologous Chondrocyte Implantation (ACI)”.

The following investigation, described in this example, was approved by Videnskabsetiske Komite for Københavns Amt and registered as # KA 99148m (190201).

Procedure:

In short, two small cartilage biopsies in the weight of 1-3 mg were harvested with a Ø 2 mm steel needle from the proximal area of the femoral condyle. The standardised cartilage biopsy samples are obtained from patients e.g., during arthroscopic examination and delivered in transport medium (DMEM/F12, gentamicin sulfate [50 μg/ml (10 mmol/l)], Fungizone [2 μg/ml (2.2 μmol/l) within 24 hours at room temperature to a laboratory.

In the cell laboratory, the two cartilage sample are first washed in PBS buffer with Calcium and Magnesium ions to secure that the samples are not contaminated with blood cells or osteogenic cell lines etc. The biopsies are weighed on a micro weight scale and the length of each biopsy is additionally recorded. Before stimulating and culturing one of the two cartilage explant samples in growth medium, the two cartilage biopsies are first cut with a sharp scalpel below the tide mark to avoid bone cells or osteoid matrix material in the analysis. One of the two cartilage biopsies is used as the reference to determine the cyto- and histo-pathologic conditions of the two samples taken from the same location. The reference sample is after the final wash fixated in 10% formalin/PBS for 48 hours before histological preparations including embedding, cutting and staining with dyes such as Safranin O, Toluidine Blue, Hematoxolin & Eosin and Gentian Violet (well-known for a person skilled in the art).

The other cartilage sample is cultured 30 days (with medium replacement every 3 and 4 day) in DMEM/F12 medium with 20% Fetal Calf Serum, Gentamycin, Ascorbic Acid and fungizone as described in Example 1.

All supernatants are collected (stored in a −80C freezer) for each medium replacement for later biochemical analysis.

After 30 days of culturing, both the cell colony forming units present in the 25 cm² culture flask and the cartilage explant sample is washed once in PBS with 1 mM magnesium and 1 mM calcium and fixated 48 hours in 10% Formalin/PBS, before staining with Safranin O, Gentian Violet or other dyes takes place (well-known for a person skilled in the art).

Each cell colony-forming unit present on the 25 cm² culture flask is measured and cell colony forming units are counted and recorded. The actual numbers of chondrogenic cell colonies present and their sizes in diameter (a certain diameter of a colony correspond to a approximated numbers of cells—and cell generations) are correlated with the histo- and cyto-pathologic evaluations of the reference sample as well as the cultured cartilage explant.

All results, derived from the biochemical investigations of the supernatant, the cell assay and the histological preparations of the biopsies, are currently been analysed and investigated and will be correlated to the patients clinical data such as: Sex, age, physical activity, general medicine, nutrition's, traumas, diseases etc., in order to obtain further information of the cause(s) of the patients knee problem(s) and the general quality of the articular cartilage in the knee.

In total, 51 patients donated cartilage biopsies for the clinical research project.

The final outcome of the clinical investigations will be present during 2002/3.

### Example 8

Relative Measurements of Growth Factors and Cytokines in Fetal Calf Serum and Human Autologous Serum

Autologous growth medium enriched with recombinant growth factors and cytokines; DMEM/F12 basal medium with 5% human autologous serum (prepared from the patients blood) is enriched with human recombinant growth factors and cytokines such as TGF-beta1(0.1-20 μg/ml), IGF-1 (0.1-20 μg/ml), IGF-2 (0.1-20 μg/ml), insulin (1-100 μg/ml), EGF (0.1-20 μg/ml), FGF-B (0.1-10 μg/ml), PDGF (0.1-20 μg/ml), GM-CSF (0.1-20 μg/ml), IL-6 (0.1-20 μg/ml), IL-8 (0.1-20 μg/ml) together with vitamin D3 (0.1-10 μg/ml), ascorbic acid (50 μg/ml, Sigma), fungizone (2 μg/ml, Gibco) and Gentamycin sulphate (100 U/ml, Gibco). Titrations and enrichment of the autologous growth medium with recombinant growth factors and cytokines is adjusted to the relative concentration of these factors present in growth medium with 20% Fetal Calf Serum as described below.

An enzyme-linked immunosorbent assay (ELISA) was used to detect the relative concentrations of growth factors and cytokines in fetal calf serum versus human serum. The human serum samples were obtained from various patients undergoing autologous chondrocyte implantation.

The relative quantifications of TGF-beta1, IGF-1, IGF-II, insulin, EGF, FGF-B, PDGF, IL6, IL-8, GM-CSF in fetal calf serum and human serum were made by sandwich ELISA using monoclonal antibodies specific for the detection of the receptive growth factors and cytokines. All monoclonal antibodies were purchased from BIØDESIGN international (Saco, Me., USA).

Monoclonal antibodies raised against growth factors and cytokines were diluted in DMEM/F12 medium in a 96 well ELISA plate (NUNC immunoplate) and incubated for 24 hours at 4 C. After 24 hours of incubation, the ELISA plates were washed and blocked in DMEM/F12 medium with 0.1% Tween 20.

Human serum samples, growth medium (prepared as described in example 1) and undiluted fetal calf serum from Life Technology was additionally serial diluted and added to the monoclonal antibodies in the ELISA system. The samples were incubated in the wells for 2 hours followed with another wash procedure with DMEM/F12 medium with 0.1% Tween 20. The various growth factors and cytokines, bound to the “primary” antibodies in the wells, were further added a “second” monoclonal antibody in the last part of the sandwich ELISA system as described above and finally developed with a “third” enzyme linked monoclonal antibody (alkaline phosphatase, BIØDESIGN) raised against the secondary antibodies.

The ELISA plates were scanned in an ELISA reader and the results were dedicated to various titration curves respectively for comparing the relative concentrations of growth factors and cytokines in human serum samples, fetal calf serum and growth medium with 20% FCS.

The growth medium with 20% FCS (earlier tested against 30 human cartilage explant samples,) was used as the titration end point for obtaining a optimal concentration of growth factors and cytokines in the autologous enriched 5% growth medium. In other words is the concentration of TGF-beta1 determined in the above EILSA system is lower in a human serum sample, compared to fetal calf serum, then recombinant human TGF-beta1 has to be added (enriched) to the autologous growth medium with 5% human serum. On the other hand, if the concentration of e.g., IGF-1 is relative higher in the human serum sample compared to 20% FCS then the autologous growth medium is not enriched further with that factor.

In this way, the relative concentration of the mentioned growth factors and cytokines can be determined for all human serum samples used for culturing cells and finally, adjusted with e.g., recombinant proteins in order to reach the optimal level of these factors in the autologous growth medium respectively, for the proper and safe in vitro culturing of mammalian cells.

### Example 9

A piece of meniscus was donated from patients undergoing knee operation and the biopsy was added to PBS buffer with magnesium and calcium in a sterile tube. In the laminar hood, the tissue was cut vertical and horizontal with a sharp and sterile knife into several pieces of tissue. The small pieces of tissue were washed another time in PBS buffer before adding the explants to growth medium as described in the protocol in Example 1.

When initially placed and stimulated by growth factors in the culture medium, it took the meniscus derived immature mesenchymal cells about 2 weeks to migrate out of the explant and to settle down on the plastic surface for establishing cell colony forming units.

The mesenchymal progenitors established in this example defined colony forming units with fibroblastic appearance on the plastic surfaces. The colonies varied in sizes (diameter) and cell numbers and show phenotypic morphologies resembling fibroblasts. The individual colonies could be further propagated into large cell numbers when cloned as individual colony forming units to larger tissue culture flasks.

It is the further goal in this project, to use the colony forming units derived from the cartilage meniscus explant in autologous cell implantation methods for repair of damaged meniscus in the knee.

1. A production plant method of producing cell colony forming units in vitro from a mammalian tissue explant, comprising the steps of;
a) growing a piece of the mammalian tissue explant in a growth medium to obtain cell colony forming units from immature cells from the piece of explant, and
b) harvesting cells from one or more of the cell colony forming units for use in Autologous Cell Implantation/transplantation methods.

2. The method according to claim 1 further comprising a step of migration and selection of the immature cells from the mammalian tissue explant into the growth medium.

3. The method according to claims 1 or 2, wherein the mammalian tissue explant is selected from tissue originating from cells derived from the group consisting of mesenchymal, ectodermal and endodermal layers.

4. The method according to any of the preceding claims, wherein the mammalian tissue explant is selected from the group consisting of cartilage; bone such as, e.g., bone marrow; connective tissue; muscle tissue such as, e.g., smooth muscle tissue, heart tissue, liver tissue and skeletal muscle tissue; skin tissue such as, e.g., periosteum; mucosal tissue; brain tissue, pancreas tissue and blood vessels.

5. The method according to claim 4, wherein the mammalian tissue explant is cartilage, such as elastic, fibro, hyalin or articular hyalin cartilage.

6. The method according to any of the preceding claims further comprising a step of rinsing the piece of the mammalian tissue explant before subjecting it to step (a) in claim 1.

7. The method according to claim 6, wherein the rinsing is performed by means of a rinsing medium, such as an aqueous medium having a pH of from about 5 to about 9.

8. The method according to any of the preceding claims, wherein the whole or at least part of the mammalian tissue explant prior to subjecting it to step (a) of claim 1 is stored at a temperature of from about −180° C. to about 37° C., such as from about −180° C. to about −70° C. or from about −70° C. to about 10° C.

9. The method according to any of the preceding claims, wherein the mammalian tissue explant is obtained by means of an instrument having a sharp end portion for inserting the instrument into the tissue and a well-defined lumen for carrying an explant of the tissue.

10. The method according to claim 9, wherein the piece of mammalian tissue explant is cut or stamped out from the mammalian tissue explant.

11. The method according to any of the preceding claims, wherein the mammalian tissue explant is partial treated with one or more proteolytic enzymes prior to subjecting it to step (a) of claim 1.

12. The method of claim 11, wherein the partial treatment is performed under conditions which enable an opening up of the structure of the mammalian tissue explant and thereby facilitating diffusion of growth factors, metabolites and immature cells in and/or out of the mammalian tissue explant.

13. The method according to claims 11 or 12, wherein the partial treatment with one or more proteolytic enzymes is performed in a concentration from about 1 to about 90 U/mg of the mammalian tissue explant

14. The method according to claim 13, wherein the partial treatment with one or more proteolytic enzymes is performed in a concentration from about 1-10 U/mg of the mammalian tissue explant

15. The method according to claim 14, wherein the partial treatment with one or more proteolytic enzymes is performed in a concentration of about 1-5 U/mg such as about 2.5 U/mg of the mammalian tissue explant.

16. The method according to any of claims 11-15, wherein the proteolytic enzyme is a proteinase.

17. The method according to claim 16, wherein the proteinases are selected from the group consisting of aspartate proteinases like Cathepsin D, cysteine proteinases like Cathepsin B, L, S, K and Calpains I and II, serine proteases like neutrophil elastase, Cathepsin G and Proteinase 3 and metallo proteinases like MMP-1, MMP-2, MMP-3, MMP4, MMP-5, MMP-6, MMP-7, MMP-8, MMP-9, MMP-10, MMP-11, MMP-12, MMP13, MMP-14, MMP-15, MMP-16, MMP-17, MMP-18, MMP-19, MMP-20 and trypsin.

18. The method according to any of the preceding claims, wherein the mammalian tissue explant is pre-treated with an aqueous medium having a pH which is at least +0.5 from the pH of 7.4 (physiologic pH), such as a pH within the range of from about 4 to about 6.9 or a pH within the range of from about 7.9 to about 10.

19. The method according to any of the preceding claims, wherein the growth medium comprises one or more components selected from the group consisting of metabolites such as, e.g., carbohydrates, lipids and amino acids; vitamins; growth factors; cytokines; minerals and antimicrobials.

20. The method according to claim 19, wherein the growth medium comprises mammalian serum, such as serum from a human, domestic or racing animal including a horse or a camel.

21. The method according to claim 20, wherein the mammalian serum is autologous to the mammalian tissue explant used in the method according to any of claims 1-19.

22. The method according to any of claims 19-21, wherein the growth medium comprises of DMEM/F12 and one or more growth factors from about 1 pico g/ml to about 100 micro g/ml.

23. The method according to any of the preceding claims, wherein the piece of mammalian tissue explant is retained during the course of culturing such as, e.g., until one or more of the cell colony-forming units are harvested.

24. The method according to any of the preceding claims, wherein the cell colony-forming units are derived from immature cells selected from the group consisting of mesenchymal, ectodermal and endodermal derived cells.

25. The method according to claim 24, wherein the cell colony-forming units are cell colony-forming units comprising cells selected from the group consisting of immature cells, stem cells, prechondroblasts, chondroblasts, chondrocytes, preosteoblasts, osteoblasts, osteocytes, myoblasts, myocytes, cemetoblastst, cementocytes, odontoblasts, odontocytes, ameloblasts, amelocytes, fibroblasts and fibrocytes.

26. The method according to any of the preceding claims comprising a further step of analysing and identifying the cells of the colony forming units.

27. The method according to any of the preceding claims further comprising a step of contacting the cell colony forming units with a release medium which enables release of the cells of the cell colony forming units from the tissue culture flask.

28. The method according to claim 27, wherein the release medium is an aqueous medium which optionally comprises one or more enzymes selected from the group consisting of proteinases such as trypsin.

29. The method according to claims 27 or 28, wherein the release medium is a buffer such as a PBS buffer without divalent metal ions.

30. The method according to any of the preceding claims further comprising a step of subjecting the piece of mammalian tissue explant to electromagnetic induction.

31. The method according to any of the preceding claims further comprising a step of enriching the mammalian tissue explant and/or the growth medium in the tissue culture flask with growth factors and/or cytokines.

32. The method according to any of the preceding claims further comprising establishing a continuous and/or pulsed delivery of growth medium, components of the growth medium or other agents to the tissue culture flask.

33. The method according to claim 32, wherein the tissue culture flask has an inlet and an outlet end portion for the continuous and/or pulsed delivery and wherein the continuous and/or pulsed delivery is established in such a manner that a difference in pressure is obtained between the inlet and the outlet ends.

34. The method according to any of the preceding claims further comprising a step of rinsing and/or culturing the harvested cell colony forming units with a medium containing at least an amount of a fluid obtained from a mammal.

35. The method according to claim 34, wherein the mammalian fluid is autologous to the piece of mammalian tissue explant cultured according to any of the preceding claims.

36. The method according to any of the preceding claims for the production of cell colony forming units in vitro from a mammalian tissue explant, comprising means for i) application of a piece of the mammalian tissue explant to a tissue culture flask, ii) application of a growth medium to the tissue culture flask, iii) growing cells migrating from the piece of mammalian tissue explant into the growth medium, and iv) application of a release medium to the tissue culture flask.

37. The method according to claim 36 further comprising means for continuous or pulsed delivery of growth medium, release medium and/or one or more factors selected from the group consisting of metabolites such as, e.g., carbohydrates, lipids and amino acids; vitamins; growth factors; cytokines; minerals and antimicrobials.

38. Cells obtainable by the method claimed in any of the preceding claims.

39. A composition comprising cells according to claim 38 and a carrier.

40. The composition according to claim 39, wherein the cells and the carrier are autologous to the mammal.

41. The composition according to claims 39 or 40, wherein the cells and carrier are autologous to the mammal, and the carrier is serum.

42. The composition of claim 39-41, wherein the carrier further contains matrix components.

43. Use of cells according to claim 38 or a composition according to claims 39-42 for use in medicine.

44. Use of cells according to claim 38 for the manufacture of a pharmaceutical composition for the treatment of tissue disorders.

45. The use according to claim 44 for the treatment of cartilage and/or bone disorders in mammals.

46. The use according to claims 44 or 45 for the treatment of cartilage and/or bone disorders in humans or in domestic or racing animals including horses and camels.

47. A transportation kit for collecting a mammalian tissue explant of a mammal, the kit comprising an instrument as defined in claim 9 for collecting a mammalian tissue explant from a mammalian tissue and a transportation container for preserving the mammalian tissue explant and, optionally, instructions for the use of the instrument.

48. The transportation kit according to claim 47, wherein the collected mammalian tissue explant is employed in a method according to any of claims 1-37.

49. The transportation kit according to claim 47 or 48, wherein the kit further comprises a blood sample tube for collecting an autologous blood sample from the mammal.

50. The transportation kit according to any of claims 47-50 for collecting a mammalian explant for use in a biochemical assay for the determination of DNA, RNA and/or protein.

51. A delivery kit comprising at least a first and a second container, the first container comprising cells according to claim 38 and a carrier and the second container comprising cartilage and/or an interface membrane.

52. A method for the autologous treatment of a mammal suffering from a tissue or tissue related disorder, the method comprising administering to the mammal in need thereof cells according to claim 38 or a composition according to any of claims 39-42.

53. The method according to claim 52, wherein the composition administered comprises autologous material including autologous serum from the same mammal.

54. The method according to claim 52 or 53 for the autologous treatment of a mammal suffering from a tissue or tissue related disorder selected from the group consisting repair of hyalin articular cartilage defects in joints, repair of hyalin/fibrous cartilage defects of the intervertebral discs, repair of larynx defects related to hyalin/fibrous cartilage, remodeling of connective tissue containing elastic cartilage used in plastic surgery methods, repair of defects bone structures related to osteoarthritis, osteoarthrosis, osteoporosis, defect bone structures do to complicated fractures and athrophic pseudo arthrosis, repair of insufficient jaw bone structure for instance related to implantation of Titanium screw for tooth repair, treatment and repair of skin burns or other skin defects related to traumas and skin related tumors as for instance hemangiomas and malignant tumors such as melanomas, repair of the ventricular wall of the heart after infarction, repair of CNS related diseases such as Parkinson, Alzheimer, dementia, multiple sclerosis and other systemic brain diseases, repair of retinitis of different pathological origin, repair of pancreatic tissue related to diabetes type I and/or II, repair of liver cirrhosis, fatty liver and for the reconstruction of liver tissue after the removal of primary liver cancers as well as liver metastases and recreation of liver structures in children with birth defects.

55. The method according to any of claims 52-54 for the autologous treatment of a mammal suffering from a cartilage and/or bone disorder or related disorder.

56. The method according to any of claims 52-55 for the autologous treatment of a mammal selected from the group consisting of a human and a domestic and/or racing animal including a horse and a camel.

57. A diagnostic method for the determination of the biological activities of a piece of mammalian tissue explant in an in vitro culture, the method comprising the steps of
(a) growing a piece of mammalian tissue explant comprising matrix and immature cells in a growth medium in a tissue culture flask, and
(b) subjecting at least a part of the content of the tissue culture flask to an investigation to receive information of the nature of the mammalian tissue explant and/or the immature cells.

58. The diagnostic method according to claim 57, wherein the investigation is a morphological, a histochemical and/or a cytopathological investigation.

59. The diagnostic method according to claim 57, wherein the content under step (b) is analysed by biochemical analysis for the determination or DNA, RNA and/or protein.
